(19) United States
(12) Patent Application Publication
(10) Pub. No.: US 2024/0024284 A1
(43) Pub. Date: Jan. 25, 2024

(54) INDOMETHACIN PHARMACEUTICAL COMPOSITION

(71) Applicant: FULTON MEDICINALI S.P.A., Arese (IT)

(72) Inventors: Alessandro GALLI, Arese (IT); Simone SARNO, Arese (IT); Claudio BELLONI, Arese (IT); Antonio GERMANI, Arese (IT)

(21) Appl. No.: 17/869,346

(22) Filed: Jul. 20, 2022

(51) Int. Cl. A61K 31/405; A61K 9/00; A61K 47/54; A61K 9/02

(52) U.S. Cl. CPC A61K 31/405; A61K 9/0031; A61K 47/543; A61K 9/02

(57) ABSTRACT

A pharmaceutical composition contains Indomethacin. The composition is a suppository containing a hard fat as a base material.

### FIELD OF THE INVENTION

The present invention relates to a pharmaceutical composition containing Indomethacin. In particular, the composition of the invention is a suppository containing a hard fat as a base material

### BACKGROUND OF THE INVENTION

The compound (I), also used in the form of a pharmaceutically acceptable salt, known as Indomethacin, is a nonsteroidal anti-inflammatory drug (NSAID) used to help relieve symptoms of moderate to severe arthritis or gout, such as inflammation, swelling, stiffness, and joint pain.

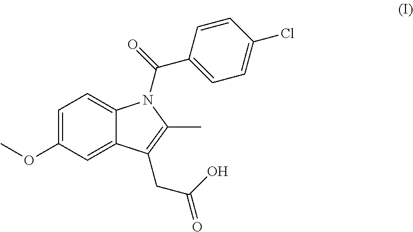 [SMILES: COc1ccc2c(c1)c(CC(=O)O)c(C)n2C(=O)c1ccc(Cl)cc1]

The present invention relates to a formulation containing Indomethacin.

### SUMMARY OF THE INVENTION

The present invention provides a pharmaceutical composition for administration to a patient in need thereof. The composition includes compound (I) or a pharmaceutically acceptable salt thereof as an active pharmaceutical ingredient, wherein said active pharmaceutical ingredient is provided in a delivery form administrable to a patient body through a body orifice, which include the rectal routes.

The present invention relates to a pharmaceutical composition comprising compound of formula (I)

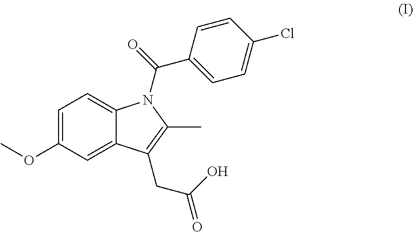 [SMILES: COc1ccc2c(c1)c(CC(=O)O)c(C)n2C(=O)c1ccc(Cl)cc1]

or a pharmaceutically acceptable salt thereof having certain desirable properties and characteristics that render these formulations suitable for administration in body orifice to relieve pain, swelling, and joint stiffness caused by arthritis, gout, bursitis, and tendonitis. The present invention further provides a storage stable pharmaceutical composition for administration to a body orifice, comprising a compound (I) or a pharmaceutically acceptable salt thereof having a purity of about 99.5% or more.

The present invention provides a pharmaceutical composition for administration to a body orifice, comprising a compound (I) or a pharmaceutically acceptable salt thereof having a purity of about 99.5% or more and less than about 0.3% each individual impurity identified at RRT about 0.71 and about 1.04 as measured by HPLC. Such impurities are mainly due to esterification of Indomethacin and its known impurities with glycerol.

The present invention further provides a storage stable pharmaceutical composition for administration to a body orifice, comprising a compound (I) or a pharmaceutically acceptable salt thereof and each individual impurity identified at RRT about 0.71 and about 1.04 not detectable as measured by HPLC.

The present invention provides a storage stable pharmaceutical composition for administration to a body orifice, comprising: a compound (I) or a pharmaceutically acceptable salt thereof having a purity of about 99.5% or more and less than 0.3% each individual impurity identified at RRT about 0.71 and about 1.04 as measured by HPLC.

Accordingly, in one embodiment, the present invention relates to a pharmaceutical composition comprising about 1% to about 25% by weight of compound (I) or of a pharmaceutically acceptable salt thereof and about 75% to about 99% by weight of a base material.

Accordingly, in another embodiment, the present invention relates to a pharmaceutical composition comprising about 30 mg to 250 mg of compound (I) or of a pharmaceutically acceptable salt thereof and about 1000 mg to 2500 mg of a base material.

In a particular embodiment, the pharmaceutical composition comprises about 1% to about 5% by weight of compound (I) or of a pharmaceutically acceptable salt thereof and about 90% to about 99% by weight of a base material.

The pharmaceutical composition described herein has advantages over prior formulations, including, for example, improved stability and less impurity as compared to currently marketed formulations.

The pharmaceutical composition described herein have slow-release profiles of compound (I) upon exposure to rectal fluid.

Yet another embodiment of the present invention is a drug delivery system that includes a pellet that changes shape or composition once inside the body and releases compound (I) once placed in the rectum. The pellet is compact in shape and size for the administration into the body. Once the pellet is placed in the rectum, or at the anal-rectal line, the pellet changes shape or composition to administer the medication contained therein. The drug delivery pellet is in the form of a suppository and can be made of a base material mixed with compound (I) or a pharmaceutically acceptable salt thereof.

### BRIEF DESCRIPTION OF THE DRAWINGS

FIG. 1 is a graph showing an impurity profile of known product

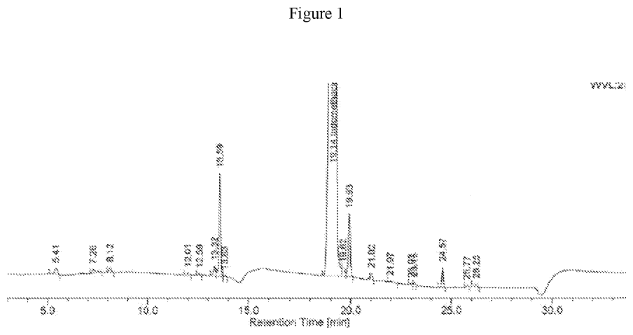

FIG. 2 is a graph showing an impurity profile of the pharmaceutical composition prepared in accordance with Example 1.

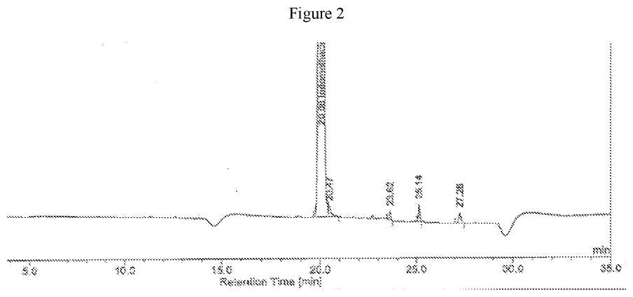

FIG. 3 is a graph showing dissolution profile of the pharmaceutical composition prepared in accordance with Example 1.

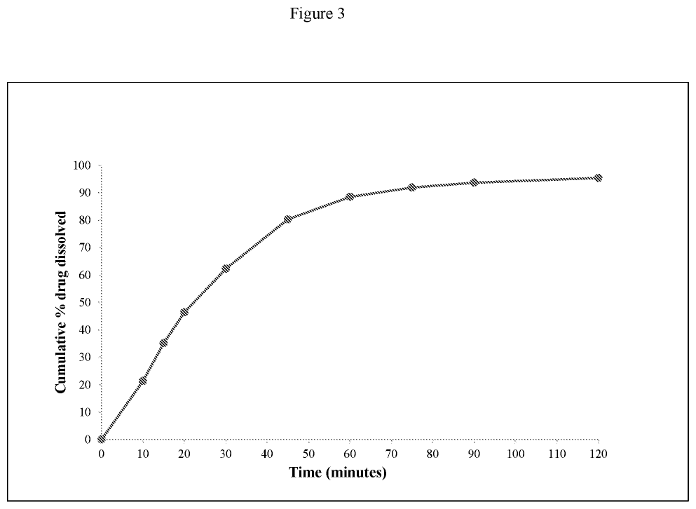

### DETAILED DESCRIPTION OF THE INVENTION

The present invention provides a pharmaceutical composition for administration to a patient body. The composition includes a compound (I) as an active pharmaceutical ingredient, wherein said active pharmaceutical ingredient is provided in a delivery form, said delivery form being administrable to a patient body through a body orifice, which include the rectal routes.

The present invention relates to pharmaceutical compositions comprising the compound of formula (I)

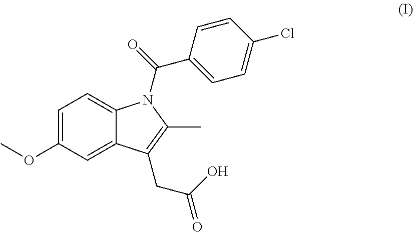 [SMILES: COc1ccc2c(c1)c(CC(=O)O)c(C)n2C(=O)c1ccc(Cl)cc1]

or a pharmaceutically acceptable salt thereof having certain desirable properties and characteristics that make these formulations suitable for administration in a body orifice to relieve pain, swelling, and joint stiffness caused by arthritis, gout, bursitis, and tendonitis. The present invention further provides a pharmaceutical composition for administration to a body orifice, comprising a compound (I) or a pharmaceutically acceptable salt thereof having a purity of about 99.5% or more and less than about 0.3% each individual impurity identified at RRT about 0.71 and about 1.04 as measured by HPLC.

The present invention further provides a storage stable pharmaceutical composition for administration to a body orifice, comprising: a compound (I) or a pharmaceutically acceptable salt thereof and each individual impurity identified at RRT about 0.71 and about 1.04 not detectable as measured by HPLC.

The present invention further provides a storage stable pharmaceutical composition for administration to a body orifice, comprising: a compound (I) or a pharmaceutically acceptable salt thereof having a purity of about 99.5% or more as measured by HPLC.

The present invention further provides a storage stable pharmaceutical composition for administration to a body orifice, comprising a compound (I) or a pharmaceutically acceptable salt thereof having a purity of about 99.5% or more and less than about 0.3% each individual impurity identified by RRT at about 0.71 and about 1.04 as measured by HPLC.

In one embodiment, the present invention provides a storage stable pharmaceutical composition comprising compound (I) and one or more pharmaceutically acceptable excipients, wherein the composition retains at least 99.5% of the compound (I) (% purity) after storage.

The storage stable pharmaceutical composition comprising compound (I) may retain at least 99.5% of the compound (I) (% purity) after storage for at least 1 month, for example, 2 months, 3 months, 6 months, at controlled temperature.

The storage stable pharmaceutical composition comprising compound (I) may retain at least 99.5% of the compound (I) (% purity) after storage for at least 1 month, for example, 2 months, 3 months, 6 months, at 30±2° C. temperature and 65% relative humidity (% RH). The storage stable pharmaceutical composition comprising compound (I) may retain at least 99.5% of the compound (I) (% purity) after storage for at least 1 month, for example, 2 months, 3 months, 6 months at 40±2° C. temperature and 75% relative humidity (% RH).

In another embodiment, the storage stable pharmaceutical composition comprises compound (I) and less than about 0.3% each individual impurity identified at RRT about 0.71 and about 1.04 as measured by HPLC after storage for at least 1 month, for example, 2 months, 3 months, 6 months, at 30±2° C. temperature and 65% relative humidity (% RH).

In another embodiment, the storage stable pharmaceutical composition comprising compound (I) does not contain impurities identified at RRT about 0.71 and about 1.04 as measured by HPLC.

Accordingly, in one embodiment, the present invention relates to a pharmaceutical composition comprising about 1% to about 25% by weight of compound (I) and about 75% to about 99% by weight of a base material.

In a particular embodiment, the pharmaceutical composition comprises about 1% to about 5% by weight of compound (I) and about 95% to about 99% by weight of a base material.

The pharmaceutical composition comprises compound (I) and a base material. The compound (I) is typically dispersed in a base material.

In one of the embodiments, the pharmaceutical composition comprises a pharmaceutically-acceptable excipient that is a base material. The base material can be a naturally occurring, semi-synthetic or synthetic compound.

The base material includes glycerides (e.g., monoglycerides, diglycerides and triglycerides). For example, the base material can include a mixture of monoglycerides, diglycerides and triglycerides, in a variety of ratios. In a particular embodiment, the base material includes triglycerides (e.g., more than 50% of the glyceride content is triglycerides). Suitable base material for use in the pharmaceutical composition may be theobroma oil, hard fats, glycerides of fatty acids, glycerol-gelatin bases, and mixtures thereof.

In the preferred embodiments, suitable hard fat bases include, but are not limited to, esterified mixtures of mono-, di- and triglycerides which are obtained by esterification of fatty acids.

In the more preferred embodiments, hard fat bases include, but are not limited to, hard fats containing a mixture of mono-, di- and triglycerides of saturated C₉₋₁₈ fatty acids. The hard fat base can comprise hard fats obtained by esterification of fatty acids of vegetable origin with glycerol, a macrogol ether containing 20 to 24 oxyethylene groups in the polyoxyethylene chain, e.g., polyoxyl-20-cetostearyl ether, and glycerides, e.g., glyceryl ricinoleate. Other suitable base materials include, but are not limited to, cocoa butter, lauric oil, beef tallow, hard fat, and any combination of any of the foregoing.

In a most preferred embodiment, hard fat basehasa melting point exactly in the range of 33.5° C. and 37.0° C., an hydroxyl value ≤10 mgKOH/g, a free fatty acid content (as oleic acid)≤0.1% and a saponification value comprised between 236 and 248 mgKOH/g. Such hard fats are for example a mixture of triglycerides C10-C18 (CAS 85665-33-4) and/or triglycerides C12-C18 (CAS 67701-26-2). They are commercially available, for example, under the name EUTECTOL M®. A preferred base material is EUTECTOL M pellets.

It has been found that the use of the above hard fat base allows to minimise the amount of impurities that may be formed over time, thus improving the storage characteristics of the pharmaceutical composition.

Accordingly, in one embodiment, the present invention relates to a pharmaceutical composition comprising about 1% to about 25% by weight of compound (I) and about 75% to about 99% by weight of a base material selected among the above mentioned hard fat materials.

Accordingly, in another embodiment, the present invention relates to a pharmaceutical composition comprising about 1% to about 10% by weight of compound (I) and about 90% to about 99% by weight of a base material selected among the above mentioned hard fat materials.

According to another embodiment, the pharmaceutical composition comprises about 1% to about 5% by weight of compound (I) and about 95% to about 99% by weight of a base material selected among the above mentioned hard fat materials.

In a preferred embodiment, the pharmaceutical composition comprises about 3% to about 7% by weight of compound (I) and about 93% to about 97% by weight of a base material selected among the above mentioned hard fat materials.

For example, the pharmaceutical composition can contain about 50 mg of compound (I) dispersed in about 1300 to about 1700 mg of a base material, most preferably a hard fat selected among the above mentioned hard fat materials.

For example, the pharmaceutical composition can contain about 100 mg of compound (I) dispersed in about 1300 to about 1700 mg of a base material, most preferably a hard fat selected among the above mentioned hard fat materials.

The total weight of the pharmaceutical composition ranges from about 1350 mg to about 1750 mg and more preferably from about 1500 mg to 1700 mg.

In additional embodiments, the pharmaceutical composition has a melting temperature in the range of about 30° C. to about 40° C., preferably about 33° C. to about 38° C.

In general, the formulations described herein are solid or semi-solid formulations. Accordingly, in various embodiments, the formulations of the present invention are suitable for use in a rectal administration to a patient body. Typically, the formulations described herein have one or more properties (e.g., melting temperature, solubility, stability) that are desirable for rectal administration. Preferably, the formulations of the invention are in the form of a suppository.

In some embodiments, the pharmaceutical composition is stable under storage conditions at a temperature in the range of about 25° C. to about 40° C. Methods for assessing stability of the pharmaceutical composition are known in the art and include, for example, the HPLC method exemplified herein below.

In other embodiments, the invention relates to a pharmaceutical composition having a weight of about 1700 mg, which comprises about 50 to about 150 mg of compound (I), and releases at least about 70% or more of the compound (I) within 90 minutes when analyzed with USP apparatus II, adopting a dissolution media comprising a buffered 0.1M phosphate solution with a final pH in the range of about 7.0 to 7.4 and maintained at 40° C.

In addition to the foregoing, embodiments of the invention are directed to the structure of the pharmaceutical composition. Yet another embodiment of the present invention is a drug delivery system that includes a pellet that changes shape or composition once inside the body and releases compound (I) once placed in the rectum. The pellet is compact in shape and size for the administration into the body. Once the pellet is placed in the rectum, or at the anal-rectal line, the pellet changes shape or composition to administer the medication contained therein. The drug delivery pellet can be made of a base material mixed with compound (I).

In other embodiments, the pharmaceutical composition is smooth torpedo-shaped. In certain embodiments, the pharmaceutical composition has a shape that allows contact between the outer surface of the pharmaceutical composition and the mucosal membrane of the rectum when the pharmaceutical composition is situated in the rectum. In other embodiments, the pharmaceutical composition releases the compound (I) upon exposure to rectal fluid. The pharmaceutical composition described herein is useful for the treatment of moderate to severe rheumatoid arthritis including acute flares of chronic disease, moderate to severe ankylosing spondylitis, moderate to severe osteoarthritis, acute painful shoulder (bursitis and/or tendinitis), acute gouty arthritis.

### Methods of Preparation of the Composition

The pharmaceutical composition of the present invention may be prepared as follows. The compound (I) is dispersed in the base material in molten form, which is then poured into a suitable mould or preformed blister made up of a material such as a PVC, polyethylene, or aluminium cavities. For example, the compound (I) may be dispersed in the base material at a temperature from about 40° C. to about 50° C.

In preferred embodiment, the compound (I) is dispersed in molten form of a hard fat and stirred for about 1-3 hours at about 40° C. to about 50° C. and poured into a suitable mould.

A pharmaceutical composition from each batch produced is preferably tested by the dissolution method for quality control. According to a preferred embodiment, a sample from each batch is tested to determine whether at least about 70% or more by weight of the compound (I) dissolves within 90 minutes.

A description of example embodiments of the invention follows.

### EXAMPLES

### Example 1

### Pharmaceutical Composition of Compound (I)

TABLE 1

| Sr. No. | Ingredient | % w/w | Quantity/Unit (mg/Unit) |
|---|---|---|---|
| 1 | Indomethacin | ~3 | 50 |
| 2 | HARD FAT NF | ~97 | 1620 |
| | (EUTECTOL M ®) | | |
| | Total | ~100 | 1670 |

### Process:

The compound (I) was dispersed in a molten Hard Fat NF (Eutectol M® pellets) at a temperature of from about 40° C. to about 50° C. and stirred for about 2 hours. The molten mass is poured into a suitable mould or preformed blister made up of a material such as a PVC, polyethylene, or aluminium cavities and packed.

### Example 2

TABLE 2

| Sr. No. | Ingredient | % w/w | Quantity/Unit (mg/Unit) |
|---|---|---|---|
| 1 | The Compound (I) | ~6 | 100 |
| 2 | HARD FAT NF | ~94 | 1570 |
| | (EUTECTOL M ®) | | |
| | Total | ~100 | 1670 |

The composition of example 1 was evaluated for Purity, Stability and Dissolution (release of the drug).

Impurity Profile by HPLC:

HPLC Method to determine purity:

A protocol for HPLC method is described below:

Preparation of Solutions

Alternate volumes of any preparation may be prepared by adjusting volumes and weights proportionately, with the exception that the weight of the standard preparations may not be reduced.

Mobile phase A: 10 g of acetic acid dissolved in 1000 mL purified water by mixing and stirring.

Mobile phase B: acetonitrile was used as solvent.

Diluent: A mixture of acetonitrile and purified water in the ratio of (70:30) % v/v was prepared as diluent.

Standard stock preparation: Transferred an accurately weigh quantity of about 50 mg of compound (I) working standard/reference standard into 50 mL volumetric flask. Added about 35 mL of diluent and sonicated to dissolve. The volume was adjusted to the mark with diluent and mix.

Standard preparation: Diluted 5.0 mL of standard stock solution to 50.0 mL with diluent and mixed. Further, diluted 2.0 mL of this solution to 100.0 mL with diluent and mix.

Sensitivity solution: Diluted 6.0 mL of standard preparation to 50.0 mL with diluent. Related compound A stock solution: Transferred an accurately weighed quantity of about 1 mg of Related compound A into 10 mL volumetric flask. Added about 7 mL of diluent and sonicated to dissolve. The volume was adjusted to the mark with diluent and mix.

System suitability solution: Transferred an accurately weigh quantity of about 1 mg of Related compound B into 100 mL volumetric flask, add 2.0 mL of Related compound A stock solution, 200 μL of standard stock solution and about 70 mL of diluent; and sonicated to dissolve. Make volume up to the mark with diluent and mix.
Column: Gemini C6-phenyl, (150 mm×4.6 mm; 3 μm)
Wavelength: 254 nm
Flow rate: 1.0 mL/minute
Injection volume: 20 μL
Column temperature: 40° C.
Vial thermostat temperature: 20° C.
Needle wash: Acetonitrile
Run time: 35 minutes

Procedure: Separately injected mobile phase (Gradient blank), diluent, sensitivity solution, system suitability solution, standard solution, placebo preparation and sample preparation into the chromatograph and record the chromatograms. Measure the responses.

Calculated the percentage of specified degradation products by using following formulas:

$$\begin{matrix}\% of specified = \\ degradation products\end{matrix} \frac{AT}{AS} \times \frac{WS}{DS} \times \frac{DT}{WT} \times \frac{P}{100} \times \frac{Average \\ Weight}{L . C .} \times R R F \times 100$$

Where,
AT=Peak area of specified degradation products in sample injection
AS=Peak area of compound (I) in standard injection
WS=Weight of compound (I) working standard/reference standard taken in mg
WT=Weight of sample taken in mg
DS=Dilution of standard preparation
DT=Dilution of sample preparation
L.C.=Label claim in mg (50 mg)
RRF=Relative response factor
P=Percentage purity of compound (I) working standard on as is basis/Declared percentage purity of reference standard

Calculate the percentage of any unspecified degradation products by using the following formulas:

$$\begin{matrix}\% of any unspecified = \\ degradation products\end{matrix} \frac{AT}{AS} \times \frac{WS}{DS} \times \frac{DT}{WT} \times \frac{P}{100} \times \frac{Average \\ Weight}{L . C .} \times R R F \times 100$$

Where,
AT=Peak area of any unspecified degradation products in sample injection
AS=Peak area of compound (I) in standard injection
WS=Weight of compound (I) working standard/reference standard taken in mg
WT=Weight of sample taken in mg
DS=Dilution of standard preparation
DT=Dilution of sample preparation
L.C.=Label claim in mg (50 mg)
P=Percentage purity of compound (I) working standard on as is basis/Declared percentage purity of reference standard

% of Total degradation product=Sum of % of all specified degradation products+Sum of % of all unspecified degradation products

### Comparative Impurity Profile of Indocin®, 50 mg and a Pharmaceutical Composition of Example 1

TABLE 3

| | INDOCIN ®, 50 mg | Example 1 |
|---|---|---|
| Related compound A | 0.11% | ND |
| Related compound B | <0.05% | ND |
| Any individual impurity | 0.44% at RRT 0.71 | BQL |
| | 0.30% at RRT 1.04 | BQL |
| Total degradation product | 1.0% | 0.0 |
| Purity of Compound (I) in | 99% | >99.9% |
| composition | | |

BQL: Below Quantitation Limit (<0.05%)
ND: Not detected

TABLE 4
Stability Data:

| Storage | | | | |
|---|---|---|---|---|
| Condition | Impurity | Initial | 1 Month | 3 Month |
| 40 ± 2° C./ | Related | ND | BQL | ND |
| 75% RH | compound A | | | |
| | Related | ND | BQL | ND |
| | compound B | | | |
| | Any unspecified | BQL | BQL | BQL |
| | degradation | | | |
| | product | | | |
| | Total | 0.00 | 0.00 | 0.00 |
| | degradation | | | |
| | product | | | |
| 30 ± 2° C./ | Related | ND | BQL | ND |
| 65% RH | compound A | | | |
| | Related | ND | BQL | ND |
| | compound B | | | |
| | Any unspecified | BQL | BQL | BQL |
| | degradation | | | |
| | product | | | |
| | Total | 0.00 | 0.00 | 0.00 |
| | degradation | | | |
| | product | | | |

BQL: Below Quantitation Limit (<0.05%)
ND: Not detected

The release of compound (I) was evaluated using a dissolution assay.

TABLE 5
Dissolution study details
Dissolution condition

Dissolution media: 0.1M phosphate buffer pH 7.2
Apparatus: USP II (Paddle)
Volume (mL): 900
RPM: 125
Temperature: 40° C. ± 0.5° C.
Time point for release and shelf life: 90 minutes
Time point for profile at initial condition: 10, 15, 20, 30, 45,
60, 75, 90 and 120 minutes

TABLE 6

| | Cumulative % Drug dissolved at time interval in minutes | | | | | | | | |
|---|---|---|---|---|---|---|---|---|---|
| Unit | 10 | 15 | 20 | 30 | 45 | 60 | 75 | 90 | 120 |
| 1 | 22.3 | 35.2 | 46.6 | 60.8 | 78.4 | 88.5 | 91.5 | 93.0 | 95.0 |
| 2 | 19.1 | 33.5 | 44.4 | 60.1 | 79.6 | 86.9 | 91.0 | 92.7 | 93.8 |
| 3 | 17.5 | 29.7 | 44.9 | 63.2 | 74.7 | 87.6 | 89.7 | 91.6 | 95.6 |
| 4 | 23.0 | 43.0 | 51.1 | 68.9 | 85.8 | 89.8 | 92.8 | 94.1 | 94.8 |
| 5 | 24.1 | 36.8 | 48.1 | 62.2 | 79.0 | 87.4 | 90.4 | 91.9 | 93.6 |
| 6 | 25.2 | 38.0 | 49.1 | 64.3 | 82.9 | 88.7 | 92.5 | 93.9 | 94.4 |
| 7 | 18.8 | 30.6 | 41.3 | 57.9 | 79.8 | 85.5 | 90.0 | 92.3 | 94.0 |
| 8 | 12.3 | 30.9 | 44.6 | 64.5 | 84.6 | 92.1 | 95.0 | 96.3 | 97.6 |
| 9 | 32.4 | 41.4 | 50.5 | 63.9 | 77.5 | 87.5 | 92.0 | 94.5 | 96.2 |
| 10 | 10.7 | 25.2 | 40.1 | 56.0 | 77.4 | 88.7 | 93.3 | 95.6 | 96.8 |
| 11 | 24.4 | 35.7 | 46.3 | 59.7 | 84.4 | 89.9 | 91.9 | 93.9 | 96.1 |
| 12 | 25.2 | 40.6 | 49.7 | 65.5 | 79.9 | 88.9 | 92.8 | 94.5 | 96.3 |
| Mean | 21.3 | 35.1 | 46.4 | 62.3 | 80.3 | 88.5 | 91.9 | 93.7 | 95.4 |
| % RSD | 28.1 | 15.2 | 7.6 | 5.7 | 4.2 | 1.9 | 1.6 | 1.5 | 1.3 |
| Mini- | 10.7 | 25.2 | 40.1 | 56.0 | 74.7 | 85.5 | 89.7 | 91.6 | 93.6 |
| mum | | | | | | | | | |
| Maxi- | 32.4 | 43.0 | 51.1 | 68.9 | 85.8 | 92.1 | 95.0 | 96.3 | 97.6 |
| mum | | | | | | | | | |

The dissolution rates of compound (I) in the pharmaceutical composition were determined by high-performance liquid chromatography (HPLC).

While this invention has been particularly shown and described with references to example embodiments thereof, it will be understood by those skilled in the art that various changes in form and details may be made therein without departing from the scope of the invention encompassed by the appended claims of this provisional application or a later-filed non-provisional application claiming priority hereto.

What is claimed is:

1. A pharmaceutical composition for administration to a body orifice, comprising a compound of formula (I), also known as Indomethacin, 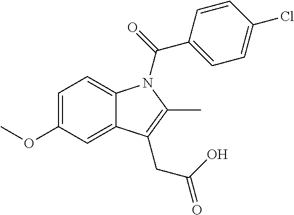 [SMILES: COc1ccc2c(c1)c(CC(=O)O)c(C)n2C(=O)c1ccc(Cl)cc1]
or a pharmaceutically acceptable salt thereof having a purity of about 99.5% or more as measured by high performance liquid chromatography (HPLC).

2. The pharmaceutical composition as claimed in claim 1, wherein the compound (I) or the pharmaceutically acceptable salts thereof have a purity of about 99.5% or more and less than about 0.3% each individual impurity identified by RRT at about 0.71 and about 1.04 as measured by HPLC.

3. A storage stable pharmaceutical composition for administration to a body orifice, comprising a compound (I) or a pharmaceutically acceptable salt thereof and less than about 0.3% each individual impurity identified at relative retention time (RRT) about 0.71 and about 1.04 as measured by high performance liquid chromatography (HPLC).

4. The pharmaceutical composition as claimed in claim 1, wherein said composition is provided in a delivery form to a body orifice through a rectal administration route.

5. The pharmaceutical composition as claimed in claim 1, further comprising a base material.

6. The pharmaceutical composition as claimed in claim 5, wherein the base material comprises a hard fat.

7. The pharmaceutical composition as claimed in claim 5, wherein said composition comprises about 1% to about 10% by weight of compound (I) and about 90% to about 99% by weight of a base material.

8. A storage stable pharmaceutical composition for administration to a body orifice, comprising a compound (I) or a pharmaceutically acceptable salt thereof having a purity of about 99.5% or more and less than about 0.3% each individual impurity identified by relative retention time (RRT) at about 0.71 and about 1.04 as measured by high performance liquid chromatography (HPLC).

9. The storage stable pharmaceutical composition as claimed in claim 8, wherein said composition retains at least 99.5% of the compound (I) (% purity) after storage for at least 3 months at 30±2° C. temperature and 65% relative humidity (% RH).

10. The storage stable pharmaceutical composition as claimed in claim 8, wherein said composition comprises from about 50 mg to about 100 mg of compound (I) and about 1600 mg of Hard Fat NF, wherein said composition releases at least about 70% or more of the compound (I) within 90 minutes when analyzed with USP apparatus II, adopting a dissolution media comprising a buffered 0.1M phosphate solution having a pH in the range of about 7.0 to about 7.4 and maintained at 40° C.

11. The storage stable pharmaceutical composition as claimed in claim 8, wherein said composition retains less than 0.3% each individual impurity identified by RRT at about 0.71 and about 1.04 as measured by HPLC after storage for at least 3 months at 30±2° C. temperature and 65% relative humidity (% RH).

12. The storage stable pharmaceutical composition as claimed in claim 8, wherein said composition comprises a hard fat base having a melting point in the range of 33.5° C. and 37.0° C., an hydroxyl value ≤10 mgKOH/g, a free fatty acid content (as oleic acid)≤0.1% and a saponification value between 236 and 248 mgKOH/g.

13. The storage stable pharmaceutical composition as claimed in claim 12, wherein said hard fat is a mixture of triglycerides C10-C18 (CAS 85665-33-4) and/or triglycerides C12-C18 (CAS 67701-26-2).

14. The storage stable pharmaceutical composition as claimed in claim 8, wherein the composition comprises a suppository.
